(19) United States
(12) Patent Application Publication
(10) Pub. No.: US 2025/0171340 A1
(43) Pub. Date: May 29, 2025

(54) THE EXPLOITATION OF PURPLE NON-SULFUR BACTERIA'S METABOLIC VERSATILITY FOR THE TREATMENT OF ZERO-NITROGEN HIGH-STRENGTH INDUSTRIAL WASTEWATER, NITROGEN PRODUCTION, AND AQUAFEED RECOVERY

(71) Applicant: Qatar Foundation for Education, Science and Community Development, Doha (QA)

(72) Inventors: Ojima Wada, Doha (QA); Hamish Mackey, Doha (QA); Gordon McKay, Doha (QA); Asifa Anwar, Doha (QA)

(21) Appl. No.: 18/951,820

(22) Filed: Nov. 19, 2024

Related U.S. Application Data
(60) Provisional application No. 63/602,832, filed on Nov. 27, 2023.

(51) Int. Cl. C02F 3/34; C02F 1/30; C12P 3/00; C12P 21/00

(52) U.S. Cl. CPC C02F 3/341; C02F 1/30; C12P 3/00; C12P 21/00

(57) ABSTRACT

A system and method of treating wastewater is provided. The method includes treating wastewater using a metabolically versatile phototrophic bacteria to recover a product. The system includes a metabolically versatile phototrophic bacteria and a specialized light filter.

### RELATED APPLICATIONS

This application claims the benefit of and priority to U.S. Provisional Patent App. 63/602,832, filed Nov. 27, 2023, the entire disclosure of which is incorporated by reference herein.

### BACKGROUND

Aquaculture, farmed seafood, has been the fastest growing food sector globally, expected to be just under 200 billion USD by 2027, resulting in higher wild fish demands and an increased reliance on terrestrial-sourced crops for aqua-feed production. Aquaculture accounts for over 50% of seafood consumed globally. However, the conventional means of producing aquaculture feed with protein sources like soybean and forage fish has contributed significantly to food and water insecurity and stress on global fisheries.

Compared with the alternatives, recovery of single-cell protein (“SCP”) from waste streams has great potential for high-quality feed within a sustainable circular economy. This is primarily because it utilizes organic waste streams, reducing the burden on scarce water, land, and mineral resources. Conventional biological wastewater treatment facilities process sludge (biomass) via energy and cost-intensive processes like landfilling or incineration. For example, a 40,000 tonnes per annum incineration plant is expected to cost 41 million USD. Diverting recovered biomass into valuable products like microbial protein will potentially result in significant financial savings as the global single-cell protein market is expected to exceed 18.5 billion USD by 2030. The potential of the recovered biomass as an alternative aquafeed was tested using larvae shrimps. Results revealed that partial replacement of commercial feed with post-wastewater treatment biomass resulted in optimal shrimp growth and significantly increased their immunity compared to the control group.

The most explored sources of SCP are yeast, fungi, and microalgae. Nonetheless, in recent times, interest in purple non-sulfur bacteria (“PNSB”) for SCP production has grown due to their metabolic versatility, ability to consume organics in diverse waste streams, high protein content, and other valuable biomolecules. PNSB has been employed to treat a wide array of industrial wastewaters, significantly reducing the organic load and upcycling nutrients. An integral factor that influences PNSB metabolism is lighting. Due to PNSB's preference for photoheterotrophy, the amount of light energy received by the light-harvesting complexes influences both biomass productivity and biomass constituents.

Typical PNSB-mediated treatment and resource recovery processes employ nutrient-rich feedstocks like agricultural wastewaters and upcycle the biomass to valuable products like microbial protein and biofertilizer for animal feed and soil enrichment purposes, respectively. However, this promising biotechnology has yet to be applicable to nutrient-deficient high-strength wastewater. PNSB's nitrogen-fixing ability has been largely unexplored for wastewater treatment and related resource recovery.

PNSB is a guild of phototrophic organisms appraised for its ability to efficiently upcycle organic pollutants and nutrients in a wide array of waste streams. Its metabolic versatility sets it apart from other organisms employed for biological wastewater treatment, as it is capable of switching between chemoautotrophy, chemoheterotrophy, photoautotrophy, and photoheterotrophy based on the culture and environmental conditions. This essentially equates to what carbon source (organic or inorganic/CO₂) and what energy source (chemical or light) the organism uses. Pollutant uptake/degradation is the most efficient in its preferred photoheterotrophic metabolism. This is because when energy is derived from illumination, PNSB can assimilate readily biodegradable organics and nutrients to its biomass at high yields (approaching 1 gram of biomass production per gram of conventional carbon consumed). In non-axenic cultures, PNSB can effectively outcompete other heterotrophs under illuminated-anoxic conditions, hindering the growth of undesirable microbes/pathogens. In addition, PNSB biomass has nutritional value based on its protein, lipids, carbohydrates, composition, and the inclusion of other valuable biomolecules such as pigments, and coenzyme Q10. This makes the recovered biomass a viable partial/whole animal feed replacement. Thus, there have been documented accounts of broadly enhancing PNSB selectivity in non-axenic cultures using NIR filters, achieving high wastewater treatment efficiency using PNSB in both axenic and non-axenic cultures, and upcycling recovered biomass for livestock feed (mostly aquaculture).

PNSB's ability to efficiently treat domestic, agricultural, and industrial waste streams has been consistently established in nutrient-sufficient wastewaters, precluding its application in nutrient-deficient waste streams. Thus, PNSB's diazotrophic abilities to anaerobically treat zero-nitrogen high-strength wastewater and produce microbial protein can be exploited. The conventional carbon (“COD”) to nitrogen ratio for typical activated sludge, microalgae, and PNSB-aided biological treatment ranges from 2:1 to 20:1. Thus, nitrogen is usually appropriately dosed for nutrient-deficient wastewater to meet treatment requirements.

However, PNSBs have been previously reported to have a diazotrophic ability via nitrogenase activity, enabling the organism to reduce N₂ to NH₃. During nitrogen-deficient conditions, the associated proteins and genes responsible for nitrogen fixation are activated and later switched off when culture conditions change (e.g. increase in culture NH₃ content or significant reduction in light intensity).

### SUMMARY

According to one non-limiting aspect of the present disclosure, a method for treating wastewater comprises using a metabolically versatile phototrophic bacteria to recover a valuable product.

In another aspect of the present disclosure, which may be combined with any other aspect listed herein unless specified otherwise, a method includes treating wastewater using a metabolically versatile phototrophic bacteria and recovering a product.

In another aspect of the present disclosure, which may be combined with any other aspect listed herein unless specified otherwise, a system includes a metabolically versatile phototrophic bacteria and a specialized light filter.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. In addition, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

Features and advantages of the present disclosure, including a process of exploiting PNSB's metabolic versatility for the treatment of zero-nitrogen high-strength industrial wastewater, nitrogen production, and aquafeed recovery, described herein may be better understood by reference to the accompanying drawings in which:

FIG. 1 is a table of the fuel synthesis water characteristics, according to an embodiments of the present disclosure.

FIG. 2 illustrates a 60 L pilot-scale with 200 W and 100 W lamps on opposite ends and 80 W LED strips beneath, according to an embodiments of the present disclosure.

FIG. 3 is a table of light characteristics and setup, according to an embodiments of the present disclosure.

FIG. 4 is a table of run characteristics, according to an embodiments of the present disclosure.

FIG. 5A illustrates TOC removal rates per average daily VSS in consecutive pilot reactor conditions (R2-C1 to R2-C4), according to an embodiments of the present disclosure.

FIG. 5B illustrates trends of VFA degradation through R2-C1 to R2-C5, according to an embodiments of the present disclosure.

FIG. 6A illustrates COD and TOC removal efficiency of pilot reactor runs, according to an embodiments of the present disclosure.

FIG. 6B illustrates biomass productivity of pilot reactor runs, according to an embodiments of the present disclosure.

FIG. 6C illustrates trends of biomass yield from run 2, conditions 1 to 5, according to an embodiments of the present disclosure.

FIG. 6D illustrates total nitrogen, phosphate, and sulfate concentrations through run 2, according to an embodiments of the present disclosure.

FIG. 7A illustrates biomass protein content for the pilot reactor at varying light intensity conditions for various runs (R1-26.4 and R2-C1-13.8), according to an embodiments of the present disclosure.

FIG. 7B illustrates biomass protein content for the pilot reactor at varying light intensity conditions for various runs (R2-C1-13.8, R2-C2-20.1, R2-C3-13.8, R2-C4-20.1, and R2-C5-13.8), according to an embodiments of the present disclosure.

FIG. 8 illustrates COD removal through the five conditions of pilot run two trials, FIG. 7A illustrates biomass protein content for the pilot reactor at varying light intensity conditions for various runs (R1-26.4 and R2-C1-13.8), according to an embodiments of the present disclosure.

FIG. 9A illustrates variations in pigment concentration through the pilot run 2 trials, according to an embodiments of the present disclosure.

FIG. 9B illustrates changes in lipid content through run 2 conditions, according to an embodiments of the present disclosure.

The reader will appreciate the foregoing details, as well as others, upon considering the following detailed description of certain non-limiting embodiments of the present disclosure.

### DETAILED DESCRIPTION

The present disclosure is generally related to the utilization of novel environmental biotechnology techniques to simultaneously treat high-strength wastewater using metabolically versatile phototrophic bacteria and recover valuable products like microbial protein and liquid nitrogen. More specifically, the present disclosure is directed to a circular economy approach where wastewater is biologically treated using a purple non-sulfur bacteria (“PNSB”)-dominated mixed culture, and biomass and treated effluent are upcycled to valuable products.

The present technology, in an embodiment, includes the use of a specific wavelength filter that can enhance the presence of a given more desirable organism over similar phototrophic organisms of the same “group” enabling more efficient treatment and biomass production, and further, the production of nitrogen from a nitrogen-deficient wastewater. From a holistic perspective, the ability to use nitrogen deficient wastewater for protein-rich biomass production is also a unique and innovative approach to treat such wastewaters, enabled through the specific organism selection.

The present disclosure addresses challenges associated with wastewater treatment, inorganic nitrogen synthesis, and food security, potentially relieving the cost and energy burden associated with conventional processes. For water treatment, a means of anaerobically treating high-strength wastewater with phototrophic bacteria-dominated mixed culture under zero-nitrogen and non-axenic conditions is proposed. This has the potential to reduce treatment expenses costs associated with aeration and nitrogen dosing are removed. This is particularly valuable as aeration is associated with the highest expenditure/energy consumption in conventional wastewater treatment facilities. Due to zero blower/aerator requirements, energy consumption associated with anaerobic digestion has been estimated to be 24.4 kWh/person equivalent, while aerobic systems use an average of 50.9 kWh/person equivalent. Additionally, this is advantageous as conventional anaerobic systems are associated with significant health and safety issues due to the release of combustible gases like methane during treatment. In this system, the out-competition of methanotrophs by PNSB ensures flammable gases are not byproducts of the treatment process.

The present technology employs a circular economy approach to simultaneously achieve wastewater treatment, inorganic liquid nitrogen production, and single-cell protein production at reduced energy and financial cost and that can be replicated in almost every part of the world (particularly in arid and tropical regions) due to the organism's versatility and universal availability of solar energy. The presently disclosed wastewater treatment technique can be applied in industries that produce zero-nitrogen or N-limited wastewaters, such as gas-to-liquid plants, chemical industry plants making polymers, solvents etc., wineries and breweries, and certain food-stuff industries. The present technology can potentially bring industries closer to achieving their carbon emission reduction goals, as well as reduce the cost of operation.

The feasibility of recovery aqua-feed bioproducts using fuel-synthesis process water (“FSPW”) as feedstock and by exploiting PNSB has been studied under varying light conditions. The results revealed that PNSB cultured in zero-nitrogen feedstock had over 40% protein content, contained substantial lipids and pigments, and reduced FSPW organics by over 70%. The light intensity significantly impacted biomass constituents and treatment efficiency.

The feasibility of using waste streams and domestic wastewater on a large scale can be largely dependent on its pretreatment requirements, heavy metal contamination, presence of pathogenic microbes, ash content, and foreign pollutants like pesticides, pharmaceutical drugs, and their metabolites.

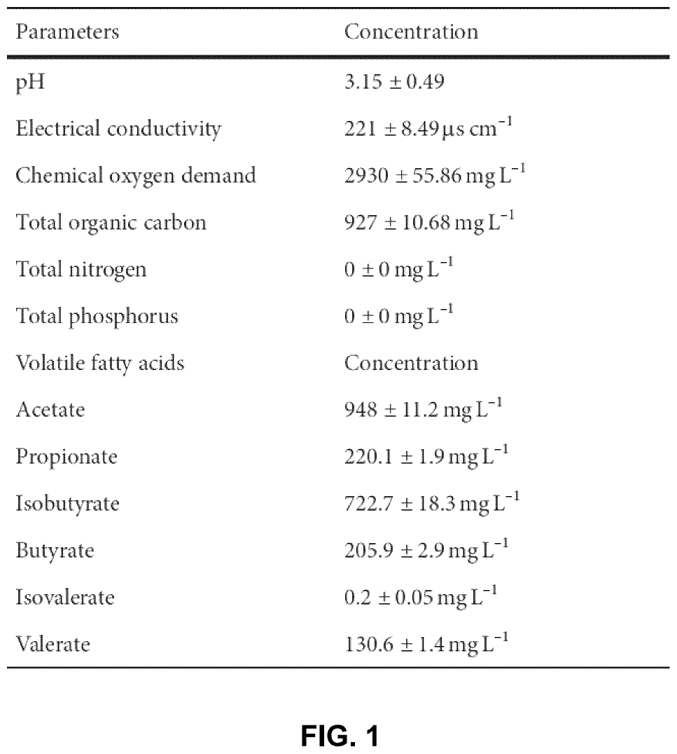

In one experiment, the potential of recovering SCP from FSPW via PNSB treatment under nitrogen-limited conditions was explored with varying light intensities. FIG. 1 is a table that summarizes the wastewater characteristics utilized in the experiment. FSPW used in the study was from a gas-to-liquid plant in Qatar. It had no detected nitrogen or phosphorus, an acidic pH (2.8-3.5), chemical oxygen demand (“COD”) around 3.1-4.6 g L⁻¹, total organic carbon (“TOC”) from 0.91 to 1.3 g L⁻¹, and total dissolved solids of <0.1 g L⁻¹. FSPW mainly consisted of shortchain organics like acetate, butyrate, isobutyrate, propionate, and valerate, accounting for over 75% of the COD. FSPW characterization of over 100 volatile and semi-volatile compounds in the USEPA 8060B and 8060C methodology also revealed that the wastewater contained negligible toxic inhibitory organics like o-xylene, toluene, benzene, and benzene derivatives. The process water was devoid of toxic heavy metal.

A non-axenic culture previously cultivated on FSPW and enriched with PNSB (predominantly Rhodopseudomonas sp.) was used for the experiment. A mixed culture was preferred to account for the practicability of operation in non-sterile outdoor settings. The initial inoculum concentration for all trials was around 50 mg L⁻¹.

The feedstock mainly consisted of undiluted FSPW (90% volume), which served as carbon substrate, along with 100 mg L⁻¹ KH₂PO₄, 81 g L⁻¹ NaHCO₃, ATCC trace mineral (10 mL L⁻¹), and ATCC vitamin (10 mL L⁻¹) supplements. The feedstock was devoid of nitrogen. The experiments were conducted in a 65 L reactor to examine the treatment and resource recovery biotechnology on a large laboratory scale. The reactor was made of polypropylene, with a working volume of 60 L and a dimension of 56 cm in width, 39 cm in thickness, and 42 cm in height. Two standing stirrers provided continuous mixing at 180 rpm. The reactor was tightly strapped at the edges to minimize oxygen transfer. The pilot reactor underwent two runs under different light intensity conditions. Runs and conditions are referred to as RX-CY-W, where X is the run, Y is a period of intra-run condition, and W is the volume-standardized light intensity.

Run 1 (R1-26.4) was performed at a constant volume-standardized light intensity of 26.4 W m⁻² L⁻¹ for 14 days without renewing the substrate. At the end of run 1, 90% of the suspended and settled biomass was decanted using a pump. Reactor walls were also scrubbed to remove biofilms. The reactor was then replenished with the same feedstock for the second run, and light conditions were changed with time.

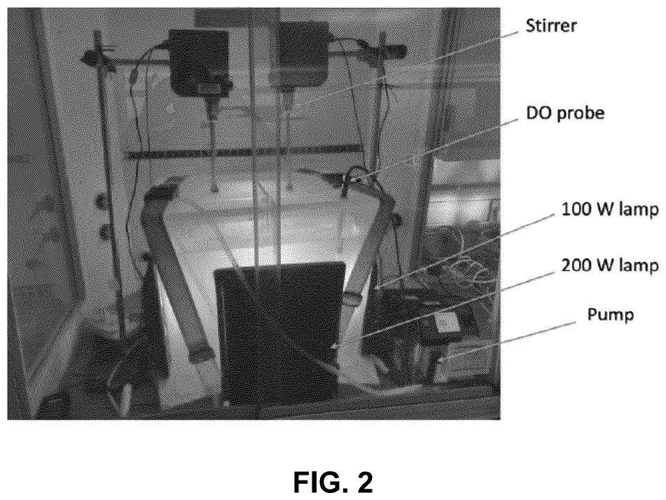

The first intra-run condition (R2-C1-13.8) was at a constant volume-standardized light intensity of 13.8 W m⁻² L⁻¹ for ten days, while the second to fifth intra-run conditions (R2-C220.1, R2-C3-13.8, R2-C4-20.1, and R2-C5-13.8) were performed at interchanging light intensities of three days each. The substrate was not renewed during both runs because organic degradation was slow due to nutrient limitation. The changes in light intensities allowed examination of photon energy on wastewater treatment, biomass yield, and recovery of SCP and pigments. A schematic of the reactor setup is depicted in FIG. 2.

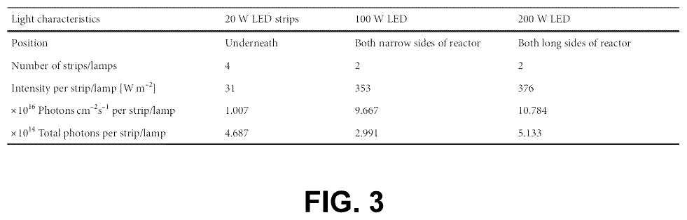

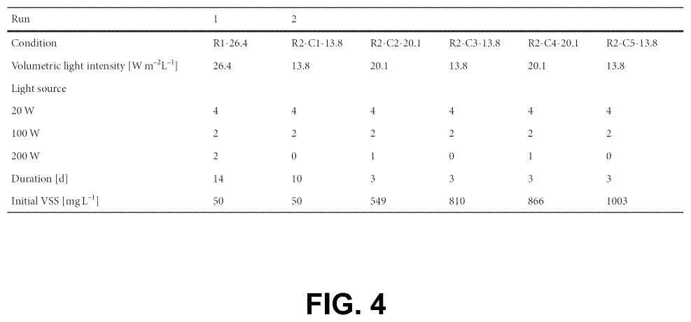

FIG. 3 provides details about light characteristics and setup, while FIG. 4 gives information about the conditions, which were changed over five periods during run 2.

Data on oxygen saturation and temperature were obtained in real time via a dissolved oxygen (DO) probe (Jenco Instruments) fixed at the top of the reactor. Spectral irradiance was measured using a Black-Comet UV-VIS spectrometer (StellarNet Inc) during reactor operation while an Ocean HDX (Ocean Insight) was employed to obtain light irradiance profiles. Parameters like pH, ORP, and temperature were measured on a ThermoFisher Scientific multiparameter meter (Orion Star A321). The optical density of the culture was monitored with a Spark multimode microplate reader (Tecan).

Samples for COD, TOC, total nitrogen (“TN”), VFAs, and anions were first centrifuged at 6000 g, after which the supernatant was sieved through 0.45-mm sterile cellulose acetate syringe filters (VWR). COD was measured via the closed microdigestion method, with Hach high-range COD vials (0 to 1500 mg L⁻¹). A TOC analyzer (TOC-L series with TNM-1, Shimadzu) determined TOC and TN. VFAs and anions were measured using ion chromatography (940 Professional IC Vario, Metrohm).

Biomass productivity in terms of total suspended solids (TSS) and volatile suspended solids (VSS) were measured by standard methods. Biomass-based analyses were performed after harvesting the centrifuging biomass at 10,000 g and freeze-drying for 24 h. Freeze-dried biomass was then homogenized and stored at −80° C. until further use. Following pretreatment via ultrasonic-assisted alkali extraction, biomass protein was quantified using the modified Lowry method. Biomass carotenoid and bacteriochlorophyll content were also measured spectrophotometrically using a multimode microplate reader.

Data analysis was performed by Microsoft Excel and JASP 0.14.1.0. Inferential statistics were used to determine statistically significant differences between the biomass content of the light groups and were measured using the independent sample t-test (for two groups) and one-way independent analysis of variance (for more than two groups) at a 95% confidence interval. The homogeneity of variance was confirmed using Levene's test and differences among groups by the Bonferroni posthoc test.

Upon incubation, the DO present in the culture was swiftly consumed within 12 h for R1-26.4 and within 26 h for R2-13.8 creating an anaerobic-light environment conducive to PNSB growth. The DO levels remained undetectable through the trials. This DO removal trend has also been reported in other studies and been attributed to consumption by aerobic/facultative anaerobic microbes. Evidence of this is seen from the microbial mat at the reactor headspace, whose upper layer is likely dominated by aerobic or facultative heterotrophic bacteria and the lower layer dominated by purple anoxygenic phototrophs. In all the trials, the initial culture pH and ORP were around 7 and −50 mV, increasing and decreasing to 9.3 and −214 mV, respectively, at the trial end. The temperature also ranged from 22° C. to 24.1° C.

Among the different pilot conditions, R2-20.1 had the highest removal rates (2.1±0.13 mg TOC L⁻¹ h⁻¹), while R1-26.4 and R2-13.8 trial groups had removal rates around 1.3 mg TOC L⁻¹ h⁻¹.

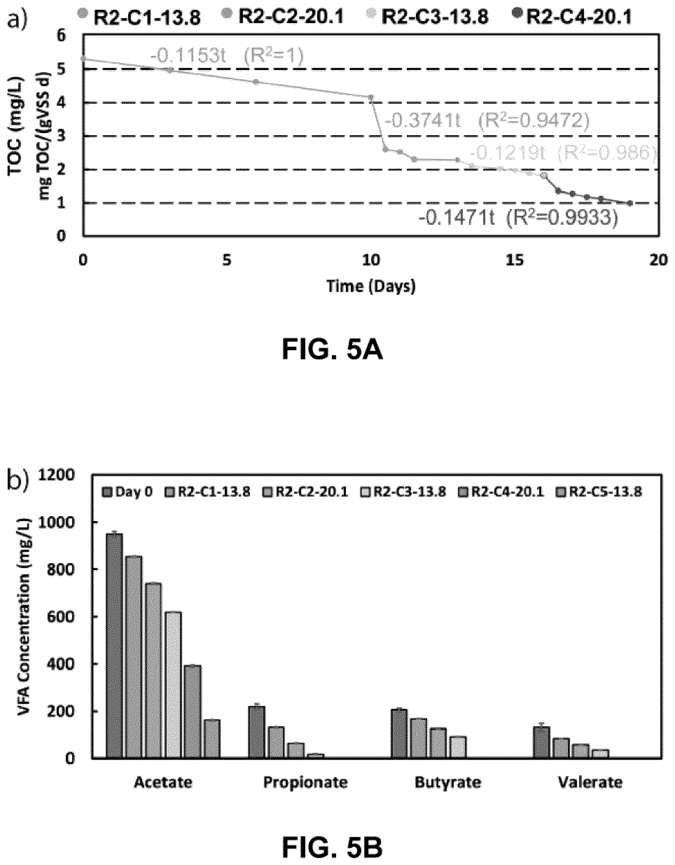

Linear equations representing the TOC removal rates per average VSS are presented in FIG. 5A, while FIG. 5B shows corresponding trends of VFA degradation. The significantly higher removal rate (p<0.005) exhibited by the R2-20.1 conditions, when compared to the higher R1-26.4 and lower intensity R2-13.8 conditions, is similar to other studies. One study found the optimum intensity for a PNSB strain (Afifella marina ME) strain grown in 112 media was 32.6 W m⁻², with lower and higher intensities leading to significantly less biomass productivity and proteolytic activity.

It is important to note that optimal light intensity greatly depends on multiple factors like the PNSB strains present, reactor size and volume, light source, and others. In a study that examined the impact of light intensity on acetate and nutrient removal by a PNSB mixed culture, treatment efficiency was highest at 350 W m⁻² and reduced at lower intensity levels (87.5-262.5 W m⁻²).

In another study, light intensity above 32 W m⁻² was found to adversely impact COD removal in chicken slaughterhouse wastewater treated with Rhodopseudomonas faecalis WS308 and Rhodopseudomonas palustris WS502. This indicates that at certain high intensities photoinhibition occurs due to light stress, while at dimmer intensities little energy is provided for metabolism. Hence, each system needs to optimize culturing conditions based on local conditions and materials. In this study, zones of inhibition were noticed on the light path of the 200 W (376 W m⁻²) and 100 W (353 W m⁻²) flood lights, while there was substantial biofilm formation over the 20 W (31 W m⁻²) LED strips' light path. This provides insights for potential biofilm production applications.

Besides nitrogen and light intensity being limiting factors for biomass growth and treatment efficiency, light attenuation is also suspected to be a contributory factor. For phototrophs, this occurs when the biomass density increases with time, thereby hindering light (energy) from circulating through the entire reactor efficiently. A recent study that examined light attenuation in enriched PNSB cultures reported that factors like biomass concentration, reactor configuration, and biomass pigment concentration significantly impact attenuation and that effective penetration in dense cultures is approximately 5 cm only. It was recommended that flat-panel reactors are used as opposed to wider systems to ensure adequate light penetration.

In this study, a similar pattern was seen as light penetration reduced over time. At the start of run 2, the light intensity at the center of the reactor, with a spectroradiometer held vertically from the top, was about 1.9 W m⁻² and reduced to about 0.06 W m⁻² at the end of condition 1. A similar inversely proportional relationship was replicated in condition 2. Under subsequent conditions, irradiance was no longer detected at the center due to biomass growth.

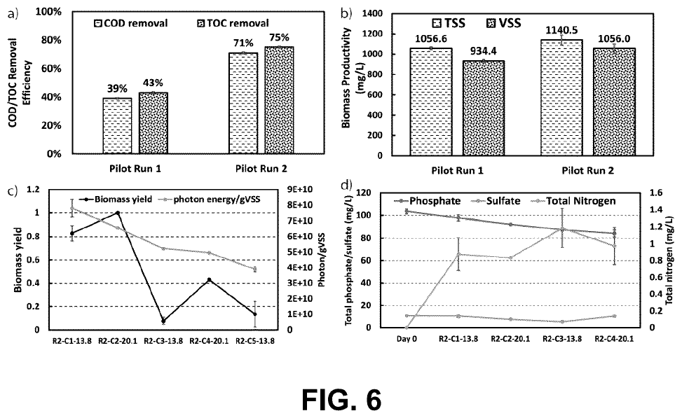

Overall, the COD removal efficiency was reasonable given light and nitrogen-limited conditions. Though taking 21 days, COD and TOC removal efficiencies in run 2 were over 70% (FIG. 6A). The biomass productivity of around 1 gVSS L⁻¹ obtained is also quite respectable, considering the light and nutrient limitations (FIG. 6B). This is due to PNSB's ability to efficiently assimilate highly reduced electron donors like propionate and butyrate using photon energy, resulting in biomass yields close to unity. The R1-26.4 group yield was 0.77±0.02 gVSS g COD_{removed}⁻¹.

Run 2 yields were mostly dependent on the light intensity. R2-C1-13.8 had a yield of 0.83, which increased to unity at the end of R2-C2-20.1. However, the yield dropped significantly to about 0.1 at the end of R2-C3-13.8 and rose substantially again by R2-C4-20.1. This trend is depicted in FIG. 6C. The increment of biomass yields as light intensity increased was rational as higher light intensity translates to more photon energy for PNSB metabolism. The total light energy per gVSS available in the reactor under conditions 3, 4, and 5 (R2-C3 to R2-C5) was significantly less than the earlier conditions due to a constantly increasing biomass concentration. This explains to some extent why conditions 3 (R2-C3-13.8) and 5 (R2-C513.8) had low yields. Moreover, the peak nitrogen concentration was around 1.2 mg L⁻¹, the phosphate removal efficiency was around 19%, while the sulfate concentration remained fairly constant (FIG. 6D).

The feasibility of SCP recovery is highly dependent on the biomass protein content. Microbial biomass, considered a protein alternative, typically consists of 30-80% protein per dry biomass weight. In this study, a protein content as high as 43% was obtained. Similar values have been reported in studies that utilized agricultural and synthetic wastewater as feedstock, indicating FSPW's feasibility as a potential substrate for PNSB-based resource recovery. Overall, the lower light intensity groups had substantially higher protein content than the higher light-intensity groups. The protein content for R1-26.4 (27.5±0.3%) was significantly (p<0.001) lower than the protein content for R2-C1-13.8 (43.4±0.7%).

A similar result was obtained in a study examining the impact of light intensity on Rhodobacter sphaeroides' biomass composition. Though not statistically significant, groups exposed to the irradiance of 3 and 5 W m⁻² had higher protein content than those exposed to 10 and 100 W m⁻². Another study that examined the influence of light intensity on biomass characteristics of an aerobic anoxygenic phototroph also reported a significantly higher protein content at lower intensities (11-33 W m⁻²) compared to higher intensities (87-435 W m⁻²). All these indicate that biomass protein productivity in anoxygenic phototrophs like PNSB could potentially be increased by limiting light exposure. Given that light penetration is one of the major constraints in bioreactor design for phototrophic systems, this finding can assist in reducing SCP production costs.

The potential recovery of SCP from nitrogen-deficient feedstock enhances the practicability of this biotechnology. Even in the absence of nitrogen, a protein content as high as 43% was achieved under nitrogen-limited conditions. A comparable biomass protein content was obtained in a similar nitrogen-deficient study. Usually, nitrogen-deficient conditions are considered for biopolymer and hydrogen production. In these instances, the culture starts with a low nitrogen concentration and then begins to accumulate biopolymers/produce H₂ when N levels are depleted. However, in this study, PNSB were cultured at zero nitrogen levels and grew with significant biomass protein content due to its possession of the nitrogenase complex. This is a feature in addition to illumination under near-infrared light that can be used to enrich non-axenic cultures for PNSB as nitrogenase enzymes are highly expressed under oxygen-limited conditions.

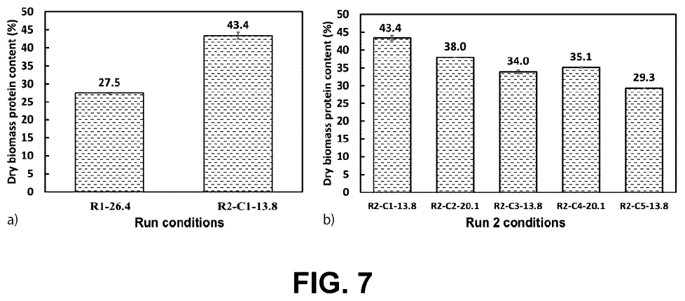

The steady decline of biomass protein content in run 2 of the pilot (p<0.05) as cultivation progressed from condition 1 to 5 (FIG. 7B) was possibly due to the accumulation of non-protein cellular constituents like cell debris, minerals, lipids, polyhydroxyalkanoates (“PHAs”), and pigments. For example, the ash content increased from 3.5±0.5% (R2-C1-13.8) to 6.3±0.9% (R2-C2-20.1) and 9.1±0.2% (R2-C3-13.8). Also, lipid and ash increased from 19.3±0.3% and 2.3±0.3% in R2-C4-20.1 to 25.3±1% and 7.4±0.1% in R2-C5-13.8. Another reason for the decline, especially by conditions 4 and 5, could be the depletion of favorable organics like propionate, butyrate, valerate, and other unidentified organics.

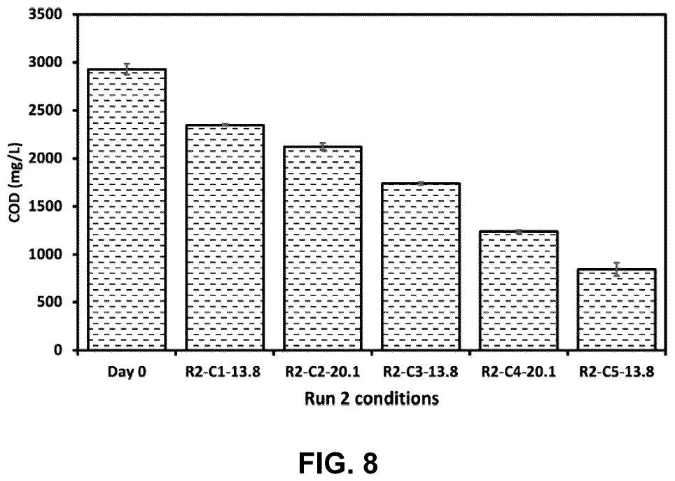

The steady decline in organics is illustrated in the COD removal trends (FIG. 8). As seen in FIG. 5B, cells under conditions 4 and 5 were mostly dependent on acetate, with its consumption increasing in condition 4 and 5 while TOC and TOC removal remained relatively constant. PNSB prefer to grow in mixed VFA substrates. One study reported a significant boost in growth rate when Rsp. Palustris, Rba. Sphaeroides, and Rhodopseudomonas ruburum were cultured in a VFA mix compared to acetate.

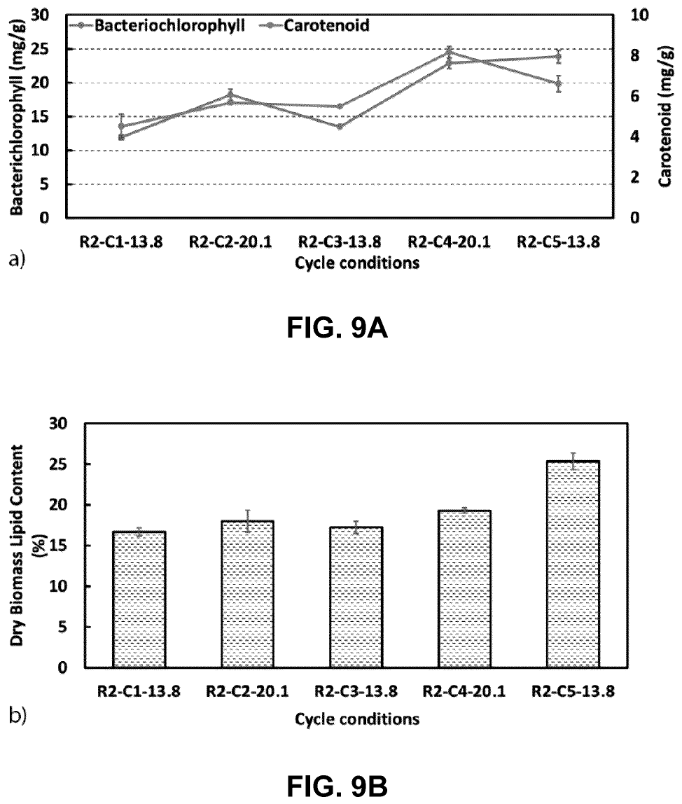

Pigments are of great value and are commonly used as food additives, coloring agents, antioxidants, and cosmetic ingredients. Recovering carotenoid-containing PNSB biomass for SCP potentially increases the feed value. This is due to the beneficial effect of carotenoids on the immune system. PNSB harvests energy using light-harvesting complexes comprised of carotenoid and bacteriochlorophyll. Therefore, PNSB's pigment content often correlates directly with the amount of energy captured. FIG. 9A indicates that the pigment concentration of PNSB with time is dependent on the light intensity. Conditions with higher intensities (R2-20.1 W m⁻²L⁻¹) had higher pigment concentrations than conditions with lower intensities (R2-13.8 W m⁻²L⁻¹). Other studies have also reported similar trends. In addition, a significant portion of the dry biomass content consisted of lipids (FIG. 9B). PNSB has been reported to consist of nutritionally beneficial lipid components like mono- and poly-unsaturated fatty acids and 5-aminolevulinic acid, potentially enhancing the feed value.

Substantial PNSB-based SCP production in zero-nitrogen feedstocks is demonstrated. This creates a premise for other nutrient-deficient wastewater to be exploited for upcycling nutritionally beneficial biomolecules. In addition, the higher biomass protein content at lower light intensities is economically and environmentally advantageous as it reduces energy requirements. The dependence of biomass yield on light intensity and available photon energy provides valuable insights into providing optimal culture conditions for treatment and resource recovery. Therefore, further experimentation is required to optimize culturing conditions.

By anaerobically treating the high-strength zero-nitrogen wastewater phototrophically, the present disclosure also provides a means to double the treatment efficiency by employing a specialized light filter to select for a more efficient organic-degrading class of microbes.

Further to this, the phototrophic organisms of interest are able to produce significant quantities of inorganic liquid nitrogen while simultaneously treating the wastewater. The production of nitrogen-containing effluent makes treated water from the present disclosure more valuable than conventional treated water utilized for agriculture. Nitrogen is an integral nutrient required by farmlands. Global demand for fertilizer nitrogen was around 110 million metric tonne in 2021 and is anticipated to increase both in demand and cost in the coming years as nitrogen is regarded as an inelastic product. Thus, treated wastewater effluent has high fertigation properties, potentially reducing the need for synthetic inorganic nitrogen fertilizers on farmlands. This inexpensive and circular economy approach to producing liquid nitrogen reduces the energy and cost burden associated with conventional production processes. In addition, the greenhouse gas offset from conventional synthetic production of inorganic nitrogen for farmlands and recovered biomass utilized as a partial replacement for aquafeed can be monetized into carbon credits.

Employment of PNSB for wastewater treatment has comparative advantages over these alternatives due to its ability to treat high-strength wastewater under high C:N ratios, adaptability/versatility under diverse environmental conditions, and absence of aeration-related energy/cost requirements. In addition, the resultant biomass is protein-rich, with a decent amino acid profile comparable to protein sources like soybean. For instance, PNSB is preferred to soybeans as close to 80% of soybean grain produced is utilized as animal feed due to it high protein content (around 35%) and decent amino acid profile. PNSB biomass has proven to have both a superior protein content (40%-50%) and amino acid profile compared to soybean. It consists of all the essential amino acids required for humans and a wide array of livestock including shrimps, poultry, catfish. The advantage of PNSB biomass over soybean production include lower land use requirement, lower freshwater requirement, applicability in desert regions and other non-agriculturally friendly regions, and probiotic effects in animals. On the other hand, PNSB is advantageous over fertilizer. The application of synthetic inorganic nitrogen fertilizer for agricultural purposes has enormous embodied greenhouse gas due to production using the Haber-Bosch process (a process that produces ammonia by a reaction of nitrogen and hydrogen under high temperature and pressure). In fact, around 2.1% of global greenhouse gas emission is associated with synthetic nitrogen fertilizers. PNSB aims to naturally extract nitrogen from the atmosphere making it readily available in the effluent which can then be irrigated to land.

The present technology employs a circular economy approach to simultaneously achieve wastewater treatment, inorganic liquid nitrogen production, and single-cell protein production at reduced energy and financial cost. The present technology can also be replicated in almost every part of the world (particularly in arid and tropical regions) due to the organism's versatility and universal availability of solar energy. The presently disclosed wastewater treatment technique can be applied in industries that produce zero-nitrogen or N-limited wastewaters, such as gas-to-liquid plants, chemical industry plants making polymers, solvents etc., wineries and breweries, and certain food-stuff industries. The present technology can potentially bring industries closer to achieving their carbon emission reduction goals, as well as reduce the cost of operation.

The present disclosure is described below in further detail including with reference to experimental examples according to an embodiment.

A first set of experiments sought to evaluate the feasibility of biologically treating nutrient-deficient GTL process water anaerobically with a Rhodopseudomonas sp. dominated mixed culture (50 mg/L) and the possible recovery of biomass microbial protein. The wastewater had negligible total nitrogen (TN) concentration (˜10 mg L⁻¹), after amendment with buffer solution (500 mg L⁻¹), phosphate (30 ppm), and trace mineral and vitamin supplements. Four replicate batch trial groups with different nitrogen conditions were examined under continuous light (white LED at an irradiance of 13 W m⁻²) for seven days in 150 mL bottle reactors. Groups 1, 2, and 3 had the feedstock augmented with over 200 mg L⁻¹ of mainstream nitrogen sources: NaNO₃, NH₄Cl and NH₂SO₄, respectively. While Group 4 had zero nitrogen added to the feedstock. Effluent treatment quality, PNSB growth parameters, and microbial protein content were monitored across the study groups. The starting C:N ratios were 5,150:1, 21:1, 21:1, and 26:1 for Zero N, NH₄Cl, NH₂SO₄, and NaNO₃, respectively, while the starting COD was around 5 g L⁻¹.

Results indicated that NaNO₃ almost completely inhibited PNSB growth as only 3% of the COD was removed, and the starting optical density (“OD”) barely doubled after seven days. NH₄Cl and NH₂SO₄ sources were most preferred by PNSB as these groups yielded the highest COD removal efficiencies (346±10 mg L⁻¹d⁻¹ and 342±28 mg L⁻¹d⁻¹, respectively). PNSB's preference for NH4+ and adverseness for NO3/NO2 have also been previously reported. For the nitrogen-sufficient conditions, there was a strong positive correlation between nitrogen removal and biomass productivity, further underscoring the importance of nitrogen for microbial growth.

Interestingly, the zero-nitrogen groups achieved decent COD removal (152±20 mg L⁻¹d⁻¹), with OD increasing by over 6-fold and biomass productivity of 114±10 mgVSS L⁻¹d⁻¹. This implies that the performance of the zero-nitrogen group was half as efficient as the NH₄+ groups, whose biomass productivities were 236±20 mg L⁻¹d⁻¹ each. The growth curve indicated that the zero-nitrogen group had a long lag phase (at least three days) before growth rates increased. The lengthy lag phase was most probably due to the lack of a readily utilizable nitrogen source like NH₄+ and limited exposure to atmospheric nitrogen, which is a rate-limiting factor for diazotrophic metabolism. However, after 72 hours, biomass growth increased exponentially, implying that the organisms utilized diazotrophic metabolism for wastewater treatment and biomass accumulation.

Protein analysis revealed that the zero-nitrogen groups had a biomass protein content of 48.4±3.7%, which was just under the NH₂SO₄ group (50.5±3.4%). The NH₄Cl (54.1±5.1%) groups had the highest protein content. Metagenomics analysis was reflective of the pollutant removal efficiency across the groups, as Rhodopseudomonas sp. prevalence positively correlated with COD removal rate. Rhodopseudomonas sp. dominance was highest in the NH₄Cl groups (70±6%), followed by the NH2SO4 groups (66±0.5%), and then the zero-nitrogen group (56±0.1%). The NaNO₃ groups had the least PNSB prevalence (39±0.4%), further confirming that the nitrogen conditions in the culture were not favorable for PNSB. Overall, these results support the hypothesis that PNSBs could use their nitrogen-fixing abilities to promote wastewater treatment and microbial protein production. The hypothesis was further validated in subsequent experiments.

Additionally, PNSB's treatment efficiency and biomass productivity can be doubled in zero-nitrogen high-strength wastewater by enhancing Rhodobacter sp. selectivity using specialized light filters. PNSB's dominance and selectivity level in mixed cultures mostly depends on the light spectra. Species of Rhodobacter and Rhodopseudomonas are the most prevalent PNSBs reported to date. Broadly purple phototrophic bacteria's selectivity in non-sterile cultures is enhanced by exposure to light of near infra-red wavelengths (805-1035 nm) due to the presence of bacteriochlorophylls a and b as light-harvesting complexes. However, specifically selecting a genre has proven more difficult. Previous studies have reported that have utilized IR light have reported increased selectivity of Rhodobacter, Rhodocyclus, and Rhodopeudomonas in poultry and domestic wastewater in a seemingly arbitrary manner. However, Rhodopseudomonas is typically the most predominant genus in mixed cultures under non-axenic conditions. This is most probably because Rhodopseudomonas sp. is reportedly more competitive to chemoheterotrophs compared to Rhodobacter sp.

Even in instances when the visible light spectrum (400-700 nm) is employed for non-axenic mixed cultures, and there is potentially increased competition with other phototrophic microbes like cyanobacteria and microalgae, Rhodopseudomonas sp. could still dominate such cultures. This is because PNSBs have a higher organic removal rate and tolerance in high-strength wastewater than other competitive phototrophic microbes. For example, in high-strength wastewater like GTL process water, Rhodopseudomonas sp. has been reported to thrive in non-axenic mixed culture under the visible light spectrum. This was also proven in the previous experiment where Rhodopseudomonas sp. predominance was reported in both nitrogen-sufficient and zero-nitrogen conditions.

However, creating a culture condition to increase Rhodobacter sp. selectivity in non-axenic mixed cultures is highly beneficial for the wastewater treatment process. Rhodobacter sp. has a significantly higher growth rate and nutrient removal rate than PNSBs like Rhodopseudomonas palustris and Rhodospirillum rubrum. In one study, the growth rate of Rhodobacter sphaeroides was over two-fold higher than that of Rhodopseudomonas palustris when cultured in a VFA mix. Thus, tests were conducted to determine if the treatment efficiency of zero-nitrogen GTL process water could be doubled by increasing the selectivity of Rhodobacter sp. using a novel light filtering technique.

Experiments were conducted under similar conditions to the first experiment. The only difference was using a light filter by placing it over the white LED. The light filter disperses around 10% of photons in the visible light spectrum, with about 90% in the NIR spectrum (mostly between 900-1,100 nm), as opposed to natural conditions when over 50% of the photon was in the visible light spectrum and about 30% between 900-1100 nm. Furthermore, the starting C:N was 5,667:1.

At the end of the trial, metagenomics analysis revealed that PNSB selectivity was about 72% which was at par with/slightly higher than the PNSB abundance under nitrogen-sufficient conditions but with unfiltered light. Moreover, Rhodobacter sp. prevalence was about 50%, significantly increasing from dormancy (<1% in the unfiltered light group), while Rhodopseudomonas sp. prevalence was just over 22%. This proves that the novel light filter utilized not only increased the overall PNSB selectivity in the zero-nitrogen high-strength wastewater, but also increased Rhodobacter sp. prevalence in the non-axenic mixed culture. Moreover, results from the biomass productivity and organic removal rate further proved PNSB's ability to employ diazotrophic metabolism for wastewater treatment. A COD removal rate of 365±20 mgL⁻¹d⁻¹ was achieved. This was more than double the COD removal rate achieved in the zero-nitrogen group under unfiltered lights, and slightly higher than the removal rate attained in the nitrogen-sufficient group under unfiltered lights. In addition, a biomass productivity of 305±14 mgVSS L⁻¹d⁻¹ was achieved. This was about 3-fold higher than the rate achieved in the zero-nitrogen group under unfiltered lights, and 1.3-fold higher than the rate attained in the nitrogen-sufficient group under unfiltered lights. This proves that Rhodobacter sp. has faster organic degradation and growth kinetics compared to Rhodopseudomonas sp.

Furthermore, protein analysis revealed a total biomass Lowry protein content of 44±0.8%. This further affirmed the possibility of microbial protein recovery from this novel treatment process. Finally, results from TN measurement revealed that there was a gradual increment in the culture's TN levels from 0 mg L⁻¹ on Day 0, to 2.4 mg L⁻¹ on Day 5 and then 5.8 mg L⁻¹ on Day 10. This led to the hypothesis for the next experiment, which examined the possibility of exploiting PNSB's diazotrophic properties for liquid nitrogen production.

Liquid nitrogen is generated as a sidestream of PNSB diazotrophic wastewater treatment and microbial protein recovery. PNSBs have broadly been appraised for their ability to fix nitrogen, with species of Rhodobacter suspected to have comparatively higher rates of nitrogenase activity. Nitrogen fixation is only possible in anoxic conditions because nitrogenase is quite sensitive to oxygen. The diazotrophic metabolism yields ammonia in the following equation: N₂+8H→2NH₃+H₂.

PNSB's diazotrophic abilities have mostly been considered for H₂ production and crop biofertilizer application but have not been broadly explored as a source of ammonium. One of the few studies that recently explored this by co-culturing Rhodopseudomonas palustris with Bacillus subtilis in an N-free medium in microplates reported an increase in TN by the trial end. In the last experiment, a similar result was obtained. However, the possibility of recovering ammonia via phototrophic nitrogen-fixation, following wastewater treatment has not been considered, particularly on a large scale and in a mixed culture. If successfully exploited, this could create a system where renewable energy (sunlight) is used for wastewater treatment, microbial protein/animal feed recovery, and liquid nitrogen generation. Thus, an experiment was conducted to determine the feasibility of exploiting PNSB's diazotrophic abilities for both industrial wastewater treatment and the production of effluent with liquid nitrogen using a 60 L pilot reactor. Thus, a non-axenic mixed culture is produced on such a large scale.

The feedstock constituents were similar to the previous experiments. A series of batch trials were conducted in an externally illuminated 60 L pilot reactor 56 cm length by 39 cm width by 42 cm height. The culture was stirred continuously at 180 rpm using two standing stirrers. The reactor was tightly strapped at the edges to minimize oxygen transfer. The only variable that changed in alternating trials was the headspace. The first set of experiments was conducted at a 60 L working volume, while the subsequent experiment was conducted at a 40 L working volume. The reduction in working volume was made to create a headspace in the reactor, which would increase the available space for air trapped above the culture. Each batch trial was set up for 21-28 days. The main reason for the extensive trial duration compared to the bottle experiments was due to the sub-optimal culturing conditions. The 60 L was quite wide, and with such a volume of culture, light dispersal becomes a challenge, especially when biomass growth begins. The light intensity at the reactor center was around 1.9±0.1 W m⁻², which is significantly less than the PNSB's conventional light requirement (<50 W m⁻²). Thus, efficiently treating high-strength wastewater in non-axenic mixed culture in a light-limited condition depending on PNSB's diazotrophic metabolism can be considered a very long stretch. However, if successful, it indicates the process can be further optimized to make substantial environmental and economic gains.

Results from the pilot study revealed PNSB's diazotrophic abilities better. Starting at zero-nitrogen concentration, the culture's liquid nitrogen concentration in the 40 L working volume trial continued to increase with time till a peak of 32 mg L⁻¹ was achieved after 21 days. This implies that nitrogen was added to the reactor at a rate of 1.5±0.1 mg TN L⁻¹d⁻¹. Within the same duration, over 60% of COD was degraded at a rate of 121±3.1 mg L⁻¹d⁻¹. A significant drop in organic removal efficiency was anticipated due to the sub-optimal culturing conditions, compared to the bottle experiments. The biomass protein content obtained (46.8±1.0%) was comparable to the bottle studies. Metagenomics analysis revealed that PNSB abundance was about 53%, which was significantly lower than the selectivity achieved in the bottle trial (72%) using the light filters. This explains the reduced organic removal rate and further proves that light was a significant limiting factor in the pilot experiment. Within 7 days of the trial, Rhodobacter sp. was the most abundant genre, with a 26.7% prevalence, followed by Rhodopseudomonas sp. with a 25.9% predominance. By day 14, the total PNSB prevalence remained around 55% but with Rhodopseudomonas sp. accounting for 41.8% and Rhodobacter sp. accounting for 13.1%. The comparatively lower Rhodobacter prevalence and significant reduction with trial duration were also suspected of contributing to the significantly lower organic removal efficiency. The reduction in Rhodobacter prevalence with time could be due to a continually decreasing light permeability. Light intensity at the center of the reactor after 10 days was less than 0.06 W m⁻². In another study with non-axenic cultures, Rhodopseudomonas was reported to be mostly dominant at long SRT, while Rhodobacter was most dominant at short SRT. However, from experiments, the main factor that likely attributed to the replacement of Rhodobacter by Rhodopseudomonas was reduced light penetration, as earlier bottle trials maintained high Rhodobacter prevalence even after 10 days. Furthermore, the higher organic removal efficiency attributable to the prevalence of Rhodobacter could be seen with the trend of weekly COD removal. Within the first week (days 0-7) and second week (days 8-14), removal rates were 166 and 167 mg COD L⁻¹d⁻¹ respectively, while removal rates over the next 14 days were 125 mg COD L⁻¹d⁻¹ for days 15-21 and 20 mg COD L⁻¹d⁻¹ for days 22 to 28. Overall, the 40 L working volume trial had superior performance compared to the 60 L working volume trials. The COD removal rate in these trials was 91±5.4 mg COD L⁻¹d⁻¹, while TN in the reactor remained <2 mg L⁻¹. However, a similar range of microbial protein was derived (43%), indicating that the nitrogen produced was most probably just sufficient to maintain cellular metabolism. Run conditions for this experiment are shown in FIGS. 2, 5A, and 5B. From the results, it can be concluded that PNSB's nitrogen fixing abilities have been underutilized in the field of wastewater treatment and microbial protein recovery. This provides novel evidence that could potentially encourage the utilization of PNSB biotechnology for nitrogen-deficient wastewater treatment while producing SCP and a treated effluent with the potential for fertigation if slower treatment rates are acceptable.

From the earlier experiments, PNSB's metabolic versatility and ability to efficiently recover organic waste have been established. However, the potential benefits of scaling up PNSB biotechnology are limited by the economic and environmental costs associated with utilizing continuous artificial lighting. Therefore, it is integral to explore the feasibility of wastewater treatment and resource recovery on the laboratory scale using non-axenic mixed cultures and photoperiods similar to natural lightning. A few studies have examined the utilization of PNSB-enriched mixed cultures for pollutant removal and resource recovery of microbial protein using synthetic and agricultural wastewater under natural photoperiod cycles. The tradeoff in using natural photoperiod cycles reported in these studies was reduced selectivity of PNSB during dark phase metabolism (giving room for other opportunistic microbes). Thus, an experiment was conducted to hypothesize increasing PNSB selectivity and predominance simply by ensuring the culture is exposed to substantial illumination just before the trial ends.

Three test groups were run based on the lighting condition. Group 1 was run in consecutive 12-hour light and 12-hour dark cycles (12 h-L/D), group 2 was run in consecutive 12-hour dark and 12-hour light cycles (12 h-L/D), while group 3 was run with continuous illumination (24 h-L). The trials were conducted in nitrogen-sufficient feedstock by adding NH₄Cl (220 mg L⁻¹). The experiments were performed using 3 identical 1 L benchtop cylindrical photobioreactors in replicated runs (Multifors, Infors). The working volume of the reactors was 700 mL, and LED lamps (unfiltered) supplied light with peak spectra around 620 nm and an irradiance of 22 W m⁻². Each batch trial lasted for 3 days and the reactors maintained a micro-aerobic condition (<2.0% O₂ saturation) all through.

16S metagenomic results from the batch trials revealed that the 24 h-L group had just over 50% prevalence of Rhodopseudomonas sp., followed by 12 h-D/L (26%), and 12 h-L/D (16%). Even though both 12 h-D/L and 12 h-L/D groups had similar illumination hours, the PNSB predominance recorded across the groups was significantly different. The higher PNSB prevalence in the 12 h-D/L group compared to the 12 h-L/D group can be attributed to the time of sample collection. For the 12 h-D/L group, sample collection was performed at the end of the light cycle, while for the 12 h-D/L sample was obtained at the end of the dark cycle. This goes to prove that a 10% increase in PNSB selectivity can be achieved just by ensuring short phases of illumination just before harvesting. This is particularly important when biomass is to be recovered as feed constituent because the higher the PNSB prevalence, the lower the predominance of competing heterotrophs which in some cases can be toxic. Thus, in outdoor conditions, a higher end of trial selectivity for PNSB can be achieved by simply providing short phases of illumination during nighttime the day before harvesting or at the end of the daytime cycle.

As achieved in the pilot experiment, biomass from a series of diazotrophic treatment processes was recovered. Previous studies that have examined the feasibility of employing PNSB biomass for partial/whole aquafeed replacement recovered the biomass from low-strength nitrogen-sufficient wastewaters, however, this differs in that the biomass is considered as a single-cell protein source. Thus, in this disclosure, the feasibility of utilizing biomass recovered from zero-nitrogen high-strength wastewater was examined using shrimp as the animal model. Aquaculture (shrimp) feed was prepared by mixing the whole biomass at different ratios with commercial feed to identify the optimal formulation ratio to maintain the shrimp's growth and composition. This is the first report of upcycling biomass from diazotrophic-based treatment to aquaculture feed.

The feeds were formulated by combining freeze-dried biomass of purple non-sulfur bacteria (PNSB) with commercially available feed at varying proportions of 0%, 15%, 30%, 45%, and 60%, based on protein content. 2% soybean oil was used as a binder in the preparation process. An additional feed was formulated using a 30% PNSB biomass blend, which was adjusted to have an equivalent energy content compared to the commercial feed. Consequently, a comprehensive set of seven conditions (including the control group fed with commercial feed) was subjected to experimentation. The formulation of the feed was established with the assumption that the protein provided to the shrimps was maintained at a fixed ratio of 0.0228 g/g of shrimp each day. A total of 20 shrimps were allocated to tanks in order to conduct experiments under seven distinct feeding circumstances.

The initial weights for all the conditions were almost similar (0.62±0.022 g). Following the 63-day trial, it was observed that shrimps that were provided with a diet containing 30% oven-dried PNSB exhibited the highest mean weight of 4.8 g, surpassing the average weight of shrimps fed with other diets, including the commercial feed, which had an average weight of 4.1 g. Subsequently, the most favorable outcomes were observed in diets containing 30% freeze-dried biomass (4.7 g) and a 30% blend of PNSB biomass, which provided an equivalent energy content to that of commercial feed (4.5 g). Hence, it can be inferred in a general sense that the incorporation of 30% PNSB in commercial feed is conducive to promoting weight gain in shrimps compared to the whole commercial feed. The shrimps that were fed a diet containing 15% PNSB also exhibited superior performance in terms of average weight (4.3 g) compared to the control group. However, the average weight of these shrimps was still lower than that of shrimps fed with any of the diets containing 30% PNSB. The experimental results indicate that the 60% PNSB blend exhibited the lowest average weight, with an estimated value of 2.0 g. Following that, the 45% PNSB diet similarly did not result in a substantial increase in shrimp biomass (3.1 g). The findings illustrate that increasing the proportion of PNSB in commercial diets above 30% does not result in a significant increase in weight gain compared to the control group.

At the conclusion of the 63-day trial, the blend containing 15% PNSB biomass exhibited the greatest final average length of the shrimps, measuring at 69.4 mm. This was followed by diets containing 30% freeze-dried PNSB biomass, which yielded a final average length of 68.0 mm, and diet containing oven dried PNSB biomass, which resulted in a final average shrimp length of 65.5 mm. The average length values observed are greater than those of the commercial diet (63.6 mm). Shrimps that were provided with a diet consisting of 30% PNSB, which had a similar energy content to that of commercially available feed, exhibited similar outcomes to the control group. The average length of these shrimps was measured to be 62.6 mm. Nevertheless, the tank that received a diet consisting of 60% PNSB had the lowest mean shrimp length, measuring a mere 44.4 mm. The inclusion of a 45% PNSB diet also resulted in a reduced length compared to the control group, which had an average length of 50.7 mm. This finding further demonstrates that a 30% PNSB biomass replacement in commercial diets would reduce reliance on conventional feed raw materials like soybean but will also improve shrimp growth parameters when compared to the control group.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method comprising:
treating wastewater using a metabolically versatile phototrophic bacteria; and
recovering a product.

2. The method of claim 1, wherein the metabolically versatile phototrophic bacteria includes a purple non-sulfur bacteria (PNSB)-dominated mixed culture.

3. The method of claim 2, further comprising employing a specialized light filter on the wastewater.

4. The method of claim 3, wherein the light filter disperses around 10% of photons in the visible light spectrum.

5. The method of claim 3, wherein the light filter disperses a light with about 90% of the light in the NIR spectrum.

6. The method of claim 3, wherein the light filter enhances PNSB's selectivity.

7. The method of claim 1, wherein the product includes a microbial protein.

8. The method of claim 1, wherein the wastewater is zero-nitrogen high-strength industrial wastewater.

9. The method of claim 1, wherein the wastewater is nitrogen-deficient.

10. The method of claim 1, wherein the product includes liquid nitrogen.

11. A system for treating wastewater comprising:
a metabolically versatile phototrophic bacteria; and
a specialized light filter.

12. The system of claim 11, further comprising a product recovered from the wastewater.

13. The system of claim 11, wherein the metabolically versatile phototrophic bacteria includes a purple non-sulfur bacteria (PNSB)-dominated mixed culture.

14. The system of claim 13, wherein the light filter disperses around 10% of photons in the visible light spectrum.

15. The system of claim 13, wherein the light filter disperses a light with about 90% of the light in the NIR spectrum.

16. The system of claim 13, wherein the light filter enhances PNSB's selectivity.

17. The system of claim 11, wherein the product includes a microbial protein.

18. The system of claim 11, wherein the wastewater is zero-nitrogen high-strength industrial wastewater.

19. The system of claim 11, wherein the wastewater is nitrogen-deficient.

20. The system of claim 11, wherein the product includes liquid nitrogen.
